(19) United States
(12) Patent Application Publication
(10) Pub. No.: US 2015/0080764 A1
(43) Pub. Date: Mar. 19, 2015

(54) DISPOSABLE INSTRUMENT INCLUDING WORKING CHANNELS FOR ENDOSCOPY

(71) Applicant: Children's Medical Center Corporation, Boston, MA (US)

(72) Inventor: Dennis Poe, Chestnut Hill, MA (US)

(21) Appl. No.: 14/337,724

(22) Filed: Jul. 22, 2014

Related U.S. Application Data
(60) Provisional application No. 61/856,894, filed on Jul. 22, 2013.

(51) Int. Cl. A61B 1/00; A61M 25/00; A61B 17/00; A61B 7/00; A61B 1/233

(52) U.S. Cl. CPC A61B 1/00142; A61B 7/003; A61B 1/0014; A61B 1/233; A61B 17/00234; A61M 25/0084; A61M 2205/75
USPC 600/586; 600/123; 604/506

(57) ABSTRACT

The present invention provides a disposable sleeve for covering an instrument. The present invention also provides an instrument embedded in a sleeve. Also provided are a method of covering an instrument with a sleeve, a method of performing a medical procedure, a method of performing sonotubometry, a method of injecting steroids to prevent scarring, and a method of filtering cavities by using the sleeve and instrument combination.

### RELATED APPLICATIONS

This application claims priority to U.S. Provisional Application No. 61/856,894, filed Jul. 22, 2013, the contents of which are incorporated in their entirety herewith.

### TECHNICAL FIELD

The present invention relates to medical instruments and sleeves therefore. In particular, the present invention relates to sleeves covering endoscopes for co-delivering of the endoscope with other medical instruments.

### BACKGROUND

Endoscopes are surgical instruments which enable a relatively non-intrusive visual inspection of and surgery on internal body tissues, particularly including body tissues located within the digestive tract. An endoscope includes a long flexible tubular member which is inserted, for example, into the colon through the anus or into the esophagus through the mouth or the nose. The tubular insertion member of an endoscope generally includes optical fibers and/or lenses for carrying light energy into the patient and for carrying organized visual information out of the patient. The insertion member also includes an elongate cylindrical channel for inserting a surgical instrument into the patient.

To improve performance, endoscopes have been optimized to best accomplish the selected purpose. As an example, endoscopes have been optimized and refined so as to provide upper endoscopes for the examination of the esophagus, stomach, and duodenum, colonoscopes for examining the colon, angioscopes for examining blood vessels, bronchoscopes for examining bronchi, laparoscopes for examining the peritoneal cavity, arthroscopes for examining joints and joint spaces, nasopharygoscopes for examining the nasal passage and pharynx, and intubation scopes for examination of a person's airway.

The operating tip of a surgical instrument, which is inserted through the ancillary biopsy channel of an endoscope, is controlled by a surgeon who manipulates an actuator at the proximal end of the endoscope. It is often difficult to maneuver an instrument through the channel to the proximal end. In order to better maneuver the instrument, the operation can be visually monitored via the visual feedback information provided by the endoscope.

One drawback to the existing methods of using an endoscope is that the functional end of the instrument is flush with the lens of the endoscope. Because of this configuration, it is difficult to see where the functional end of the instrument is located when the endoscope is at a site of operation. In many instances it is therefore necessary to insert an additional instrument into the patient to be at the correct angle to operate. The instrument is inserted into the patient's tissue blindly, with the physician hoping that the instrument will appear in the field of vision of the endoscope. This requires the use of both of the physician's hands, one for the endoscope and one for the instrument, and requires additional physicians to aid in suction or irrigation.

Because endoscopes are expensive instruments, they are used on multiple patients and must accordingly be cleaned after each procedure. Some endoscopes have been made with metal sheaths covering the channels; these types of endoscopes, however, pose risks to the patient's health if they are not properly sterilized. Cleaning generally entails soaking at least the distal end of the endoscope's insertion member in an antibacterial and antiviral solution. In addition, the operating channels of the insertion member are flushed, preferably with an antimicrobial cleaning solution. Such cleaning procedures require substantial amounts of time. Costs are increased, not only because of the hospital personnel time involved, but also because the endoscopes are out of use for that additional time. Moreover, there is always the risk that the cleaning is inadequate and that dangerous bacteria, viruses, or prions remaining in the endoscope may be subsequently transferred to a patient. This risk cannot be ignored in the present environment of AIDS and other dreaded diseases.

Disposable sheaths for endoscopes have been made in order to minimize the contamination of the endoscope, with a new sheath being put on the endoscope for each operation. This reduces the time for sterilization and the risk of infection to each patient. For example, U.S. Pat. No. 5,536,234 to Newman describes a detachable, disposable semi-flexible sheath for insertion into a patient's body. The sheath includes a plurality of channels in which to receive an endoscope for use with suction, drainage, or irrigation. However, it is difficult to insert an instrument through the semi-flexible sheath once inside the patient's tissue.

There is therefore a need for a sheath to protect an endoscope from contamination that also allows for easy manipulation of an instrument inside the sheath. There is also a need for an instrument that can easily be seen in the field of vision of the endoscope. Finally, there is a need for an instrument and sheath combination that can enable the physician to use one of their hands to perform other functions.

### SUMMARY OF THE INVENTION

The present invention provides a disposable sleeve for covering an instrument including a body having a distal end and a proximal end, and includes an embedded instrument within the body in an instrument entrapment portion defining a longitudinal axis and at least one passageway extending substantially parallel to the longitudinal axis for receiving at least one additional instrument. The present invention also provides an instrument embedded in a sleeve including a body having a distal end and a proximal end. A sleeve is provided including a body having an instrument entrapment portion with a disposable instrument embedded therein defining a longitudinal axis, and at least one passageway extending substantially parallel to the longitudinal axis. A disposable sleeve is provided for covering an instrument including a body having a distal end and a proximal end, and includes a trough or a ridge on the body defining a longitudinal axis, an instrument fixedly attached to the trough or ridge, and at least one passageway extending substantially parallel to the longitudinal axis for receiving an additional instrument. Also provided are a methods of covering an instrument with a sleeve, performing a medical procedure, performing sonotubometry, injecting steroids to prevent scarring, and filtering cavities by using the sleeve and instrument combination.

### BRIEF DESCRIPTION ON THE DRAWINGS

Other advantages of the present invention will be readily appreciated as the same becomes better understood by reference to the following detailed description when considered in connection with the accompanying drawings wherein:

FIG. 1 is a perspective view of a sleeve of the present invention with an embedded instrument retracted;

FIG. 2 is a cross-sectional view of FIG. 1 taken along line A-A of a sleeve with one passageway showing a longitudinal axis;

FIG. 3 is a perspective view of a sleeve with an embedded instrument extended;

FIG. 4 is a cross-sectional view of FIG. 1 taken along line B-B;

FIG. 5 is a cross-sectional view of FIG. 1 taken along line A-A of a sleeve on an endoscope that is curved;

FIG. 6 is a cross-sectional view of FIG. 1 taken along line A-A of a sleeve on an endoscope showing the functional end of the disposable embedded instrument in the field of vision of the endoscope;

FIG. 7 is a cross-sectional view of FIG. 1 taken along line B-B showing a sealed distal end of the passageway;

FIG. 8 is a perspective view of a sleeve showing a second passageway with an additional instrument;

FIG. 9 is a cross-sectional view of FIG. 1 taken along line A-A of a sleeve and endoscope combination with an embedded disposable speaker for use in sonotubometry;

FIG. 10 is a cross-sectional view of FIG. 1 taken along line A-A of a sleeve and endoscope combination with an embedded fluid injection needle;

FIG. 11 is a cross-sectional view of FIG. 1 taken along line A-A of a sleeve and endoscope combination with an embedded filtration device embedded in a second sleeve;

FIG. 12 is a cross-sectional view of FIG. 1 taken along line A-A of a sleeve and endoscope combination with an embedded cautery end, ultrasound probe, or radiofrequency probe;

FIG. 13 is a cross-sectional view of FIG. 1 taken along line A-A of a sleeve and endoscope combination with an embedded knife;

FIG. 14 is a cross-sectional view of FIG. 1 taken along line A-A of a sleeve and endoscope combination with an embedded balloon shown both deflated and inflated;

FIG. 15 is a cross-sectional view of FIG. 1 taken along line A-A of a sleeve and endoscope combination with an embedded speaker with electromyographic recording electrodes, a pressure/airflow transducer, and a balloon for use in Eustachian tube studies;

FIG. 16 is a cross-sectional view of FIG. 1 taken along line A-A of a sleeve and endoscope combination with multiple embedded instruments;

FIG. 17 is a perspective view of a sleeve with a longitudinal trough and a fixed instrument;

FIG. 18 is a cross-sectional view of FIG. 17 taken along line C-C of a sleeve with a longitudinal trough;

FIG. 19 is a perspective view of a sleeve with a longitudinal ridge and a fixed instrument; and

FIG. 20 is a cross-sectional view of FIG. 19 taken along line E-E of a sleeve with a longitudinal ridge.

### DETAILED DESCRIPTION

The present invention provides a disposable sleeve for use in. covering an instrument, preferably an endoscope. It is particularly useful in endoscopic surgery.

Like structure among the several defined embodiments are indicated by primed numbers.

A “sleeve” is an enveloping structure, and is also used interchangeably with “sheath.” A sheath is used to cover and protect an endoscope and other instruments from contamination during surgery.

The term “embedded” . means inserted as an integral part of the surrounding whole. For example, an instrument is embedded in a sleeve, meaning that the instrument is inserted inside the sleeve and is an integral part of it.

The term “instrument” means a device used to facilitate work. In the present invention, the instrument is one that is used in a medical procedure. The instrument can be any suitable medical instrument, including, but not limited to, an endoscope.

The term “embedded instrument” generally refers to an instrument embedded in a sleeve that can be manipulated from outside the sleeve to perform a function inside a patient's body and is further detailed below.

The term “additional instrument” refers to an instrument that is not embedded in the sleeve and is generally freely able to be inserted and withdrawn within the sleeve, The additional instrument is preferably an endoscope, but can be any other suitable instrument, and is further detailed below.

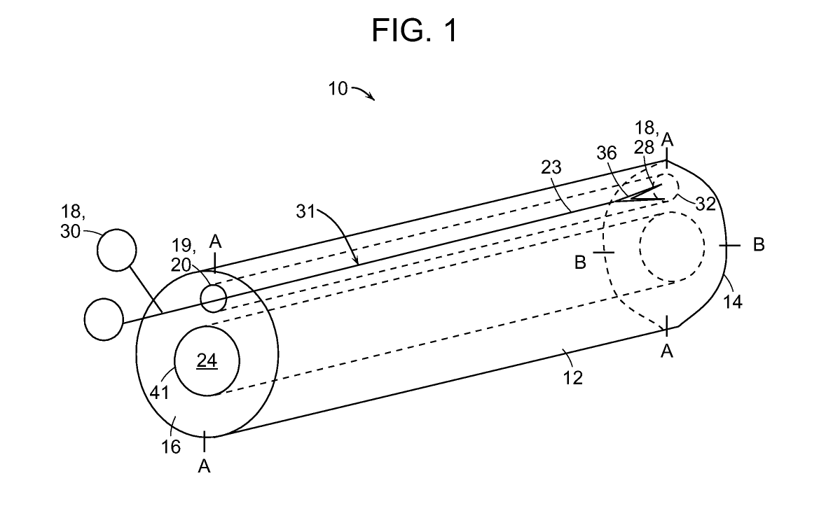

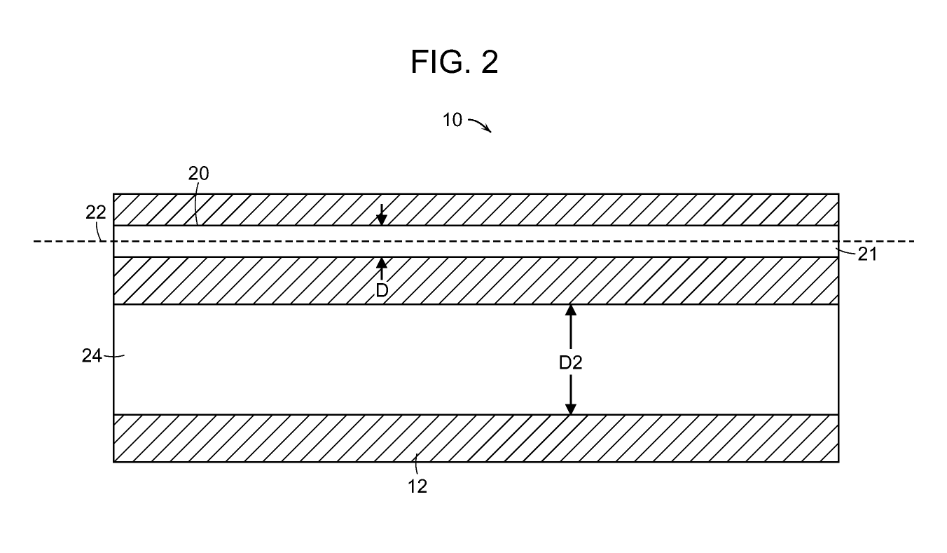

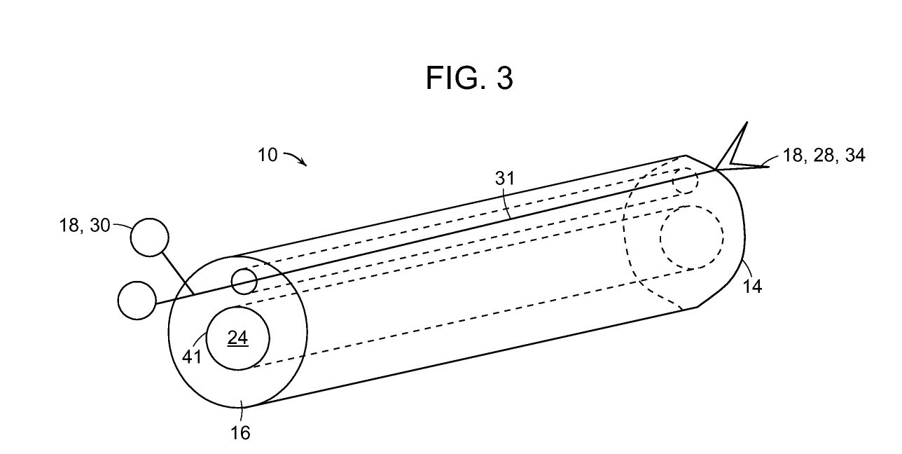

The present invention provides a disposable sleeve shown generally at 10 for covering an instrument as shown in FIGS. 1 and 3. The sleeve 10 includes a body 12 having a distal end 14 and a proximal end 16 opposite the distal end 14. The body 12 includes an embedded instrument 18 that is embedded within the body 12 in an instrument entrapment portion 20. The instrument entrapment portion 20 defines a longitudinal axis 22, as shown in FIG. 2. There is at least one passageway 24 that extends substantially parallel to the longitudinal axis 22 which receives an additional instrument 25.

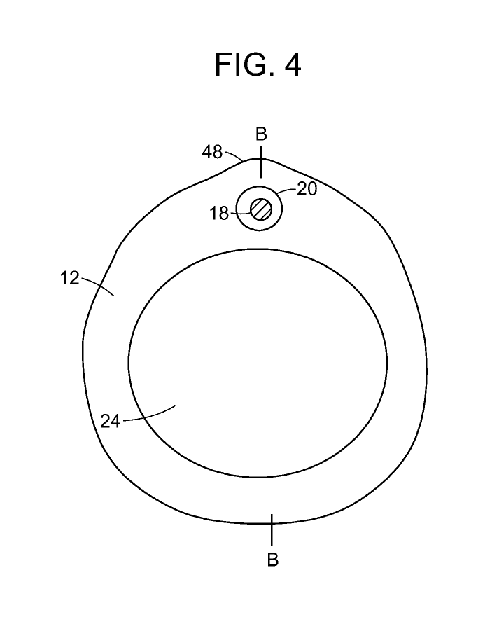

The body 12 can be formed as a thin material, but the body 12 can include thicker areas around passageways and especially around the instrument entrapment portion 20, as shown in FIG. 4. There can be a bump, protrusion, or thickened area 48 on the body 12 indicating where the embedded instrument 18 is located. The bump 48 is preferably less than a few millimeters above the surface of the body 12. The size of the bump 48 will be dependent on the thickness of the embedded instrument 18 and the type of material used for the body 12. However, the bump 48 will result in a minimal increase in the thickness of the body when compared to prior art devices.

Preferably, the body 12 is made from a semi-flexible plastic, elastomeric material, vinyl or other polymer, polytetrafluoroethylene (such as Teflon®), or any other material appropriate for the particular application. The material is preferably biocompatible and complies with Federal Drug Administration (FDA) and International Organization for Standardization (ISO) requirements, and therefore does not cause toxicity or irritate the tissue with which the body 12 comes into contact. Preferably, the material is inexpensive and disposable.

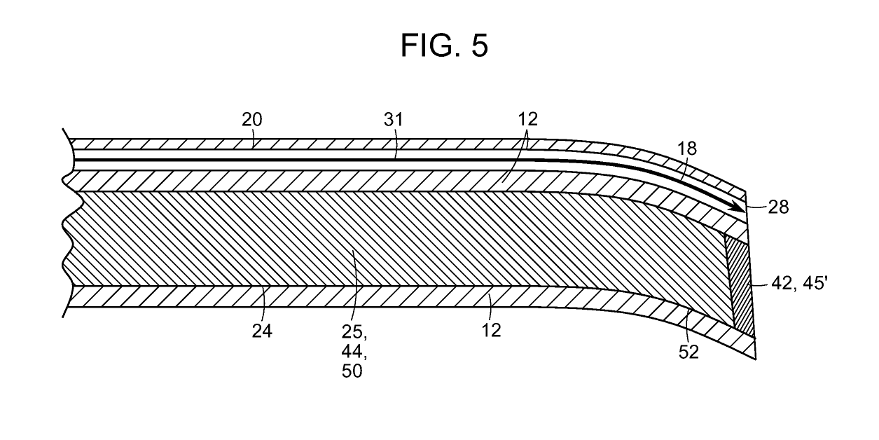

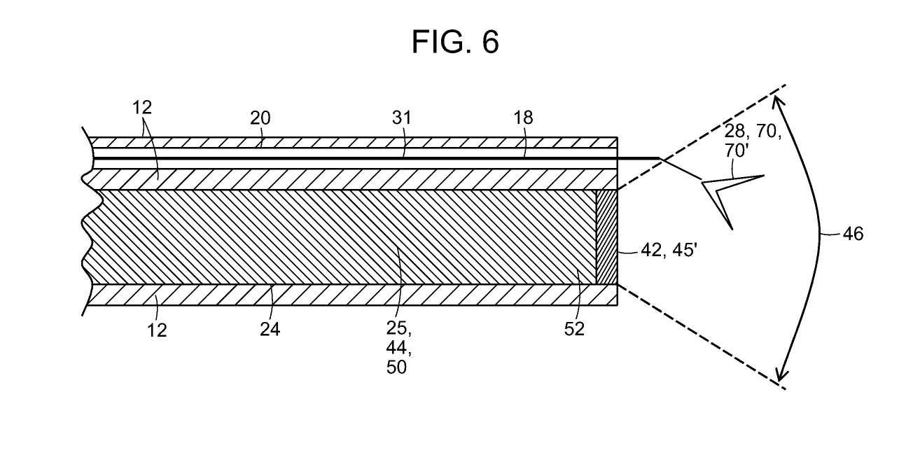

Preferably, the additional instrument 25 is an endoscope 44. Use with a flexible endoscope 44, such as a fiber optic endoscope 44, as shown in FIG. 5, requires that the sleeve to be able to move with the endoscope 44, so a flexible or semi-flexible material is preferably used, such as a flexible fiber. A flexible material also allows for easier insertion of an instrument, either an embedded instrument 18 during the manufacturing process or an additional instrument 25 during a medical procedure, especially one that does not have a constant cross-sectional area because of protrudements. Use with a rigid endoscope 44, as shown in FIG. 6, allows for a more rigid material to be used for the body 12, such as a rigid fiber or other rigid material capable of being used in this manner.

An instrument entrapment portion 20 extends between the distal 14 and proximal 16 ends of the body 12 and includes the embedded instrument 18 extending along a length of the instrument entrapment portion 20. The instrument entrapment portion 20 includes a distal end 32 and a proximal end 19. A cross-sectional diameter D of the instrument entrapment portion 20 is preferably circular; however, it can also be other shapes as required for use. For example, the cross-sectional diameter D can be a custom shape to fit a particular embedded instrument 18, The size of the cross-sectional diameter D can vary depending on the embedded instrument 18 desired to be inserted in the sleeve 10 during manufacturing. Preferably, the cross-sectional diameter D is small, and more preferably smaller than the cross-sectional diameters of other passageways 24 in the sleeve. The instrument entrapment portion 20 is not designed as a passageway itself, but is instead a place for the embedded instrument 18 to rest inside the body 12, It is also desirable to be able to maneuver the embedded instrument 18 while in the instrument entrapment portion 20, therefore the size of the cross-sectional diameter D of the instrument entrapment portion 20 preferably permits slight movement of the embedded instrument 18. Preferably, there is not much space between an inside surface 21 of the instrument entrapment portion 20 and an outer surface 23 of the embedded instrument 18 because it is not desirable to have bodily fluids escape down the instrument entrapment portion 20.

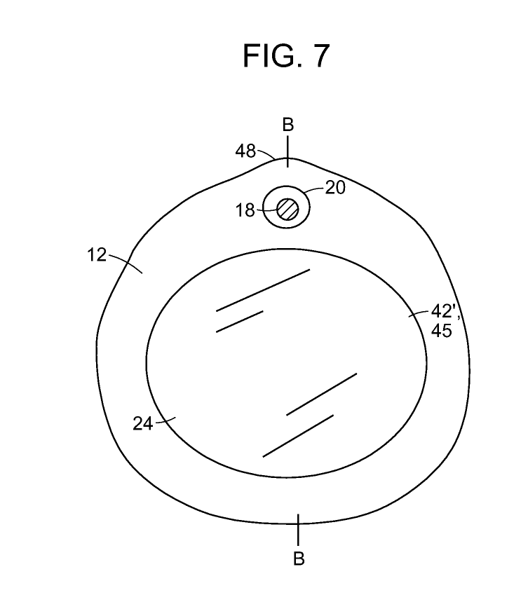

The passageway 24, in which the additional instrument 25 is inserted, also extends along the length of the body 12 of the sleeve 10 from the distal end 14 to the proximal end 16. The passageway 24 includes an open proximal end 41 and a distal end 42. Depending upon the use of the passageway 24, it can include either a closed distal end 42′ shown in FIG. 7, or an open distal end 42″, as shown in FIG. 4, For example, when an endoscope 44 is inserted into the passageway 24, it is preferred that there is a closed distal end 42′, which can be closed by an end cap 45. The end cap 45 can be integral with the body 12 or it can be made separately, with the same or different material as the body 12, and sealably attached to the body 12 to make a closed distal end 42. Furthermore, with the use of an endoscope 44, the end cap 45 is preferably an optically transparent cap 45′ so as not to block the vision of the endoscope 44. An optically transparent cap 45′ allows for endoscopic visualization in a patient when the endoscope 44 is inserted in the sleeve 10. The optically transparent cap 45′ can be a clear lens. The optically transparent material can be, but is not limited to, clarified polypropylene, polyethylene terephthalate, polystyrene, polyvinyl chloride, polycarbonate, and glass.

The passageway 24 has a cross-sectional diameter D2 like that of the instrument entrapment portion 20. The cross-sectional diameter D2 shape and size are appropriate for the particular additional instrument 25 inserted into the passageway 24 and can be variable with respect to the instrument being inserted. For example, with the endoscope 44, the cross-sectional diameter D2 is a circular shape to accommodate the elongated insertion member 50 of the endoscope 44, and the size of the cross-sectional diameter D2 is small enough so that the endoscope 44 is not moving freely inside the body 12. The sleeve preferably includes no extraneous material because during use such material can be twisted, bunched, or misaligned relative to the elongated insertion member 50. Extraneous material can also increase the diameter of the endoscope 44, which can increase pain or discomfort to the patient. The passageway 24 is also of an appropriate length to cover the additional instrument 25, and can be varied according to the additional instrument 25 being used.

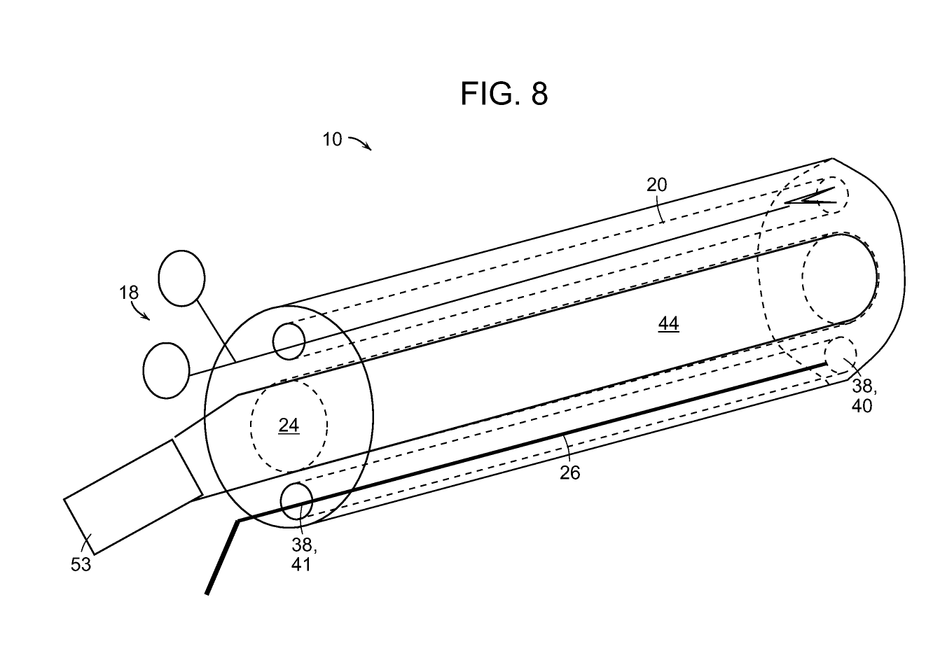

The sleeve 10 can optionally include more passageways 24 for multiple instruments extending from the distal end 14 to the proximal end 16 and substantially parallel to the longitudinal axis 22. For example, as shown in FIG. 8, a second passageway 38 can be included for the purpose of suction, drainage, or irrigation wherein a third instrument 26 is inserted through the second passageway 38. The other passageways 24 can serve any other suitable purpose.

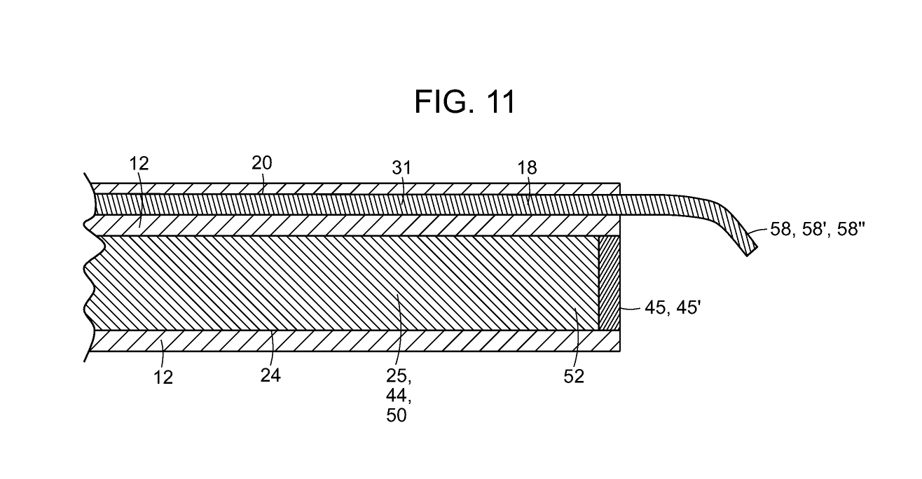

The embedded instrument 18 can be made from any suitable materials, such as a rigid or flexible material, by manufacturing methods known in the art. The embedded instrument 18 can be disposable along with the entire sleeve 10. The embedded instrument 18 can be sterilized and reused in another procedure. The embedded instrument 18 is embedded in the instrument entrapment portion 20, or in some manner fixed or fastened thereto. The embedded instrument 18 can alternatively be housed in a second sleeve 11, which fits inside the instrument entrapment portion 20 and is in some manner fixed or fastened thereto, as shown in FIG. 11.

The embedded instrument 18 includes a functional end 28 and a manipulative end 30 opposite the functional end 28. The functional end 28 is located at the distal end 32 of the instrument entrapment portion 20. The functional end 28 is used to perform part of a surgical procedure. The functional end 28 can be fully encompassed by the distal end 32 of the instrument entrapment portion 20 when inserted into tissue, or it can be fully or partially protruding outside of the distal end 32 when inserted into tissue. In other words, the functional end 28 of the embedded instrument 18 can terminate at any point along the instrument entrapment portion 20 as desired. Further, the functional end 28 can protrude from the instrument entrapment portion 20 at various lengths and angles, and can be manipulated by a telescoping movement described below.

An elongated portion 31 operatively connects the functional end 28 and the manipulative end 30, and extends along the length of the instrument entrapment portion 20. For example, the elongated portion 31 can be a hollow rod containing wires along its length connected to both the functional end 28 and the manipulative end 30 that allows for the manipulation of the functional end 28 by a physician. The elongated portion 31 can also be a chamber housing a laser. The elongated portion 31 can be stiff or flexible. A flexible elongated portion 31 can be used with the flexible endoscope 44 or additional instrument 25. The elongated portion 31 can be any other suitable design that allows for manipulation of an embedded instrument 18 from outside of the patient's tissue.

The manipulative end 30 of the embedded instrument 18 protrudes from the proximal end 19 of the instrument entrapment portion 20. The manipulative end 30 is a device that the physician can manipulate to cause the functional end 28 of the embedded instrument 18 to function. Furthermore, the manipulative end 30 can be manipulated to make the functional end 28 telescope in or out of the distal end 32 of the instrument entrapment portion 20 or to rotate the functional end 28. In FIG. 1, the embedded instrument 18 is shown with a retracted functional end position 36. In FIG. 3, the embedded instrument 18 is shown with a protruded functional end position 34. The telescoping can be accomplished by a motorized extension of the elongated member 31 when the physician presses a button on the manipulative end 30 of the embedded instrument 18. Another way to accomplish the telescoping is by manipulating a tension wire 33 inside the elongated member 31 attached to the manipulative end 30. Any other method of telescoping can be used. The telescoping ability is advantageous when the functional end 28 is sharp or has an awkward shape, so that the functional end 28 can be retracted into the distal end 32 of the instrument entrapment portion 20 in order to allow smooth movement of the sleeve 10 and additional instrument 25 combination through. tissue. When at an operating site, the functional end 28 can then be deployed out of the distal end 32 of the instrument entrapment portion 20.

One advantage of the present invention is that the functional end 28 of the embedded instrument 18 is in the field of vision 46 of the passageway 24 when deployed outside of the distal end 32 of the instrument entrapment portion 20. In other words, the functional end 28 can be viewed directly by the endoscope 44 inserted in the passageway 24. For example, the functional end 28 can protrude 1 cm past the distal end 32 of the instrument entrapment portion 20 and inwards toward the field of vision 46 at a 30 degree angle. Any suitable protruding length or angle can be used. The embedded instrument 18 being in the field of vision of the endoscope 44 of the present invention is distinguishable from other endoscopes (with or without a sleeve) with fixed instruments, such as a laser, in one of the endoscope's channels. A fixed instrument is in direct alignment with the endoscope and does not protrude, and is therefore not in the endoscope's field of vision. There are instruments in the prior art which are not fixed, such as biopsy forceps and stone grasping baskets, and can be passed through the conventional working channel of existing endoscopes to protrude into the endoscope's field of vision. However, because the instruments have to be passed through an existing working channel, they are limited in diameter. These prior art instruments are also often excessively mobile or flexible, and therefore much harder to control than the embedded instruments 18 of the present invention. Furthermore, the diameter of the embedded instruments 18 of the present invention is not limited, and the instrument entrapment portion 20 can be designed to fit the diameter of a specific embedded instrument 18. The ability of the embedded instruments 18 to protrude into the field of vision 46 solves the problem of trying to find a separately inserted instrument or maneuver it to the tissue site of the endoscope 44. In the present invention, the embedded instrument 18 follows the exact path of the endoscope 44 during insertion into tissue, therefore, once the endoscope 44 is at the operative site, the embedded instrument 18 is also at the operative site,

The embedded instrument 18 can be used for any suitable purpose such as diagnostic uses or therapeutic uses, medical uses, surgical uses, industrial uses, or forensic uses, several of which are further detailed below. The embedded instruments 18 can be connected to devices that are powered by any means, for example, hand powered tools, electrical tools, vacuum devices, positive pressure devices, and fluid movement devices. The embedded instrument 18 can also be actively or passively mobile, steerable, or have various moving parts which are active or passive. The embedded instrument 18 can be removed from the sleeve 10 during a procedure and exchanged for a different embedded instrument 18. The embedded instrument 18 can also be repositioned within the sleeve 10 to change the angle of the functional end 28.

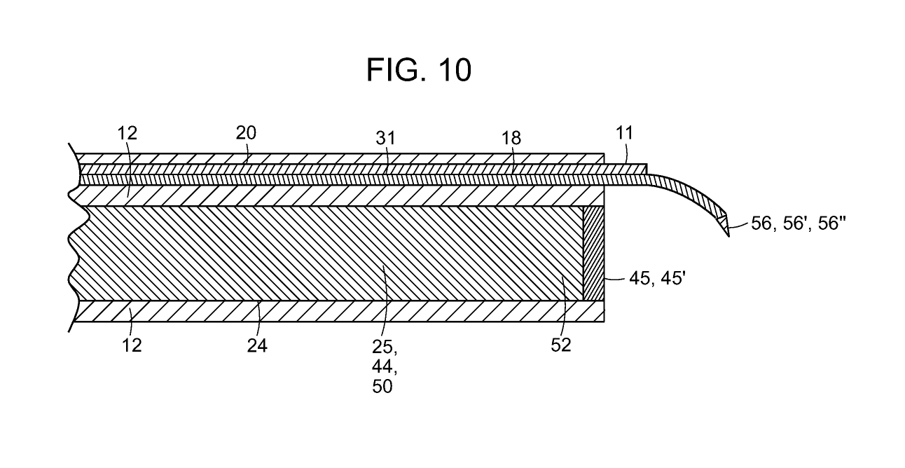

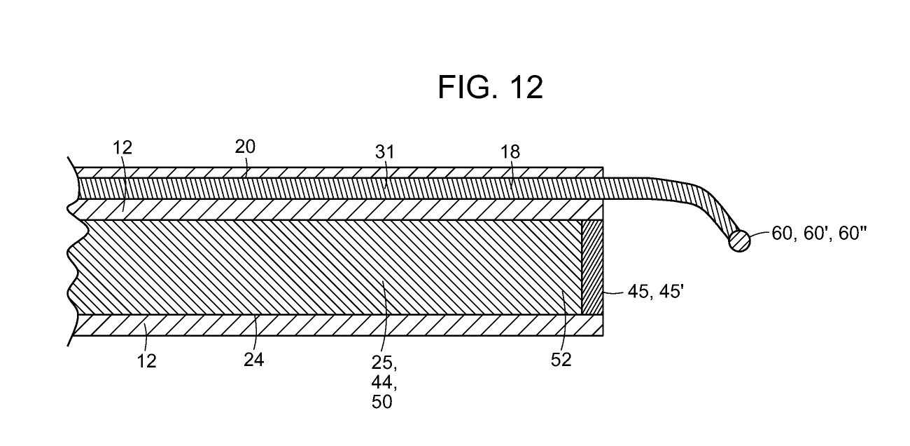

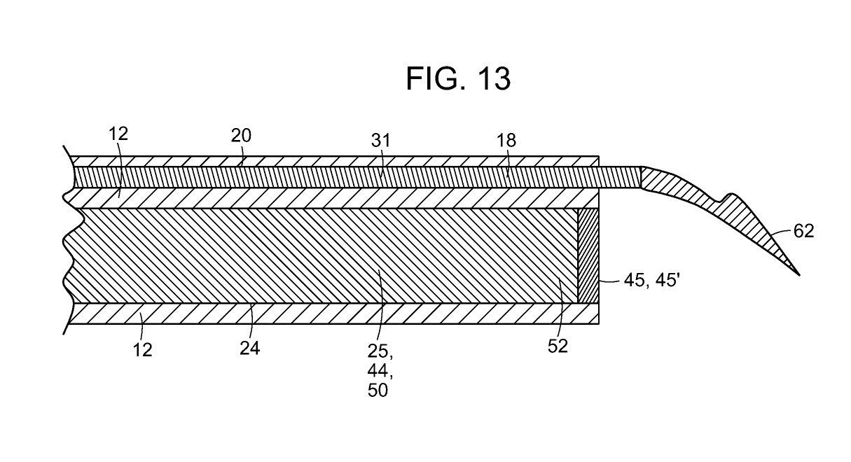

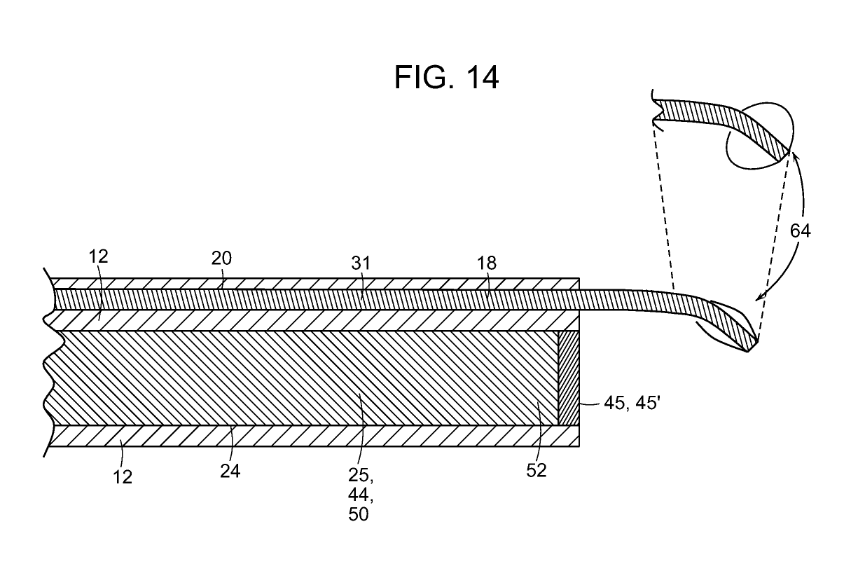

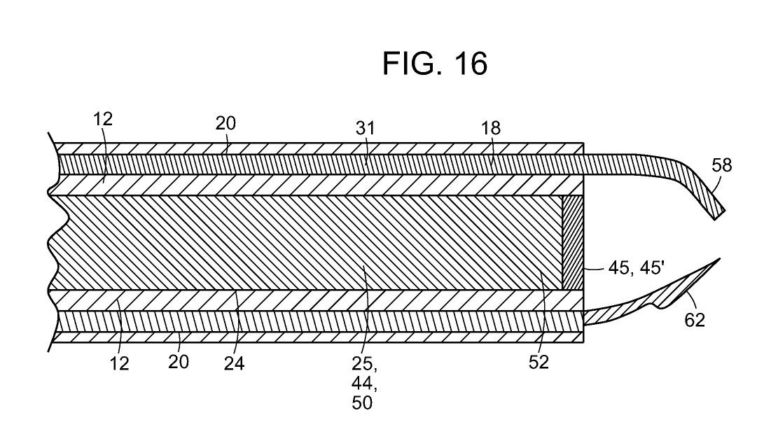

The embedded instrument 18 can be any instrument that allows for manipulation a length away from the functional end 28. For example, in endoscopic surgery, the embedded instrument 28 can be a folding catheter, a dilating probe, a test probe such as for testing electrical currents in the heart, a pressure sensing device, a fluid injector, biopsy forceps, a laser, or a needle. The embedded instrument 28 can also be a laser that can be used for various purposes such as laser surgery. The embedded instrument 18 can take on many other different forms, such as, but not limited to, an electric monopolar or bipolar cautery 60, an ultrasound probe 60′ or a radiofrequency probe 60″ shown in FIG. 12. The embedded instrument 18 can be a retractable injection needle 56, an aspiration needle 56′, or a radiofrequency needle 56″, as shown in FIG. 10. The embedded instrument 18 can be a knife 62, or any other sharp or blunt dissecting object, as shown in FIG. 13. The embedded instrument 18 can be a balloon 64, as shown in FIG. 14, which can be inflated when positioned outside of the instrument entrapment portion 20. The balloon 64 can further be used with instrumentation such as a sheath lumen, suction, diagnostic or therapeutic instruments. The embedded instrument 18 can be a measurement probe 58′ such as a transducer for pressure measurement, temperature measurement, Doppler probe, motion sensor, or spectroscopy as shown in FIG. 11. The embedded instrument 18 can be a treatment probe 58″ such as a laser, photodynamic Rx light, or radioactive seeds, also as in FIG. 11. The embedded instrument 18 can further be other sensors or stimulators such as microphones and speakers. The embedded instrument 18 can also be forceps 70, scissors 70′, or any other suitable instrument for biopsy or other surgical purpose, as shown in FIG. 6. The embedded instrument 18 can be a combination of instruments for multiple Eustachian tube studies including the speaker 54, electromyographic recording electrodes 66, a pressure/airflow transducer 68, and a balloon 64, further detailed below. Further, multiple embedded instruments 18 can be embedded in one or more instrument entrapment portions 20, such as the sleeve 10 shown in FIG. 16 with the filtration device 58 and the knife 62. Any suitable combination of instruments can be used.

The sleeve 10 can be packaged with the embedded instrument 18 already inside the instrument entrapment portion 20, so the user does not need to insert additional instruments. In order to insert an additional instrument 25 through the passageway 24, the distal end 52 of the additional instrument 25 is inserted in the proximal end 41 of the passageway 24. The sleeve 10 is slid over the length of the additional instrument 25 until the distal end 52 of the additional instrument 25 is at the distal end 42 of the passageway 24. During this process, the distal end 52 of the additional instrument 25 is aligned with the functional end 28 of the embedded instrument 18 and a proximal end 53 of the additional instrument 25 is aligned simultaneously with the manipulative end 30. The sleeve 10 can also be rotated on the additional instrument 25 so as to align the embedded instrument 18 in a desired place.

The sleeve 10 can be used with the endoscope 44 or any other suitable medical device during a surgical procedure. For example, the endoscope 44 can be inserted in the sleeve 10 as described above and then the endoscope 44 and sleeve 10 combination is inserted into a patient's tissue to the operation site. The physician can telescope the functional end 28 of the embedded instrument 18 into the field of vision 46 of the endoscope 44 (if the functional end 28 was inside the distal end 32 of the instrument entrapment portion 20) and view its position with the endoscope 44. The manipulative end 30 is manipulated so that the functional end 28 performs at least one step of the surgery. After the procedure is finished, the endoscope 44 and sleeve 10 combination can be removed from the patient's tissue. The sleeve 10 with the embedded instrument 18 are removed from the endoscope 44 and can then be disposed. The endoscope 44 is protected from contamination and is free to be used in the next procedure with minimal cleaning.

The sleeve 10 is advantageous in medical procedures because the sleeve 10 is an impermeable, disposable protective cover for the additional instrument 25, it protects against exposure of the additional instrument 25 to fluids and thus protects the patient against infection. The sleeve 10 also facilitates and/or eliminates damaging cleaning of the additional instrument 25. Another advantage of the present invention is that the sleeve 10 and endoscope 44 combination enables one of the surgeon's hands to be available. Previously, the physician used one hand for the endoscope 44 and another for a surgical instrument. This meant that an extra person was required to perform suction, drainage, or irrigation, or an endoscope holding device would be employed. However, using the sleeve 10 with an embedded instrument 18 on the endoscope 44 allows the physician to use his or her other hand to perform another procedure or hold onto another instrument. The sleeve 10 and endoscope 44 combination are coupled and steerable, so the physician knows where the embedded instrument 18 is going and can feel comfortable directing it with one hand.

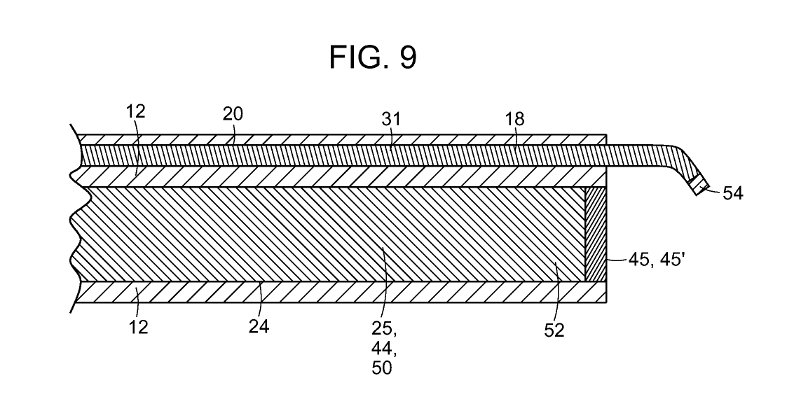

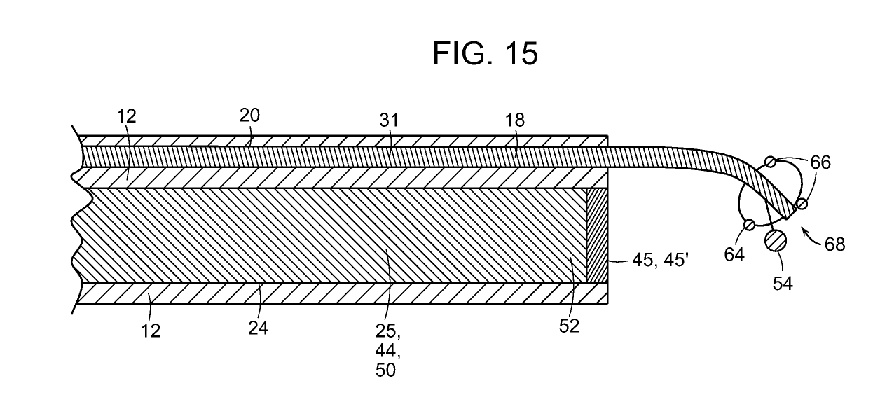

The sleeve 10 and endoscope 44 combination is an ideal instrument for performing a sonotubometry test. A sonotubometry test is done with a speaker in a patient's nostril and a microphone in an ear that listens for an increase in the speaker's tone which occurs when the Eustachian tube opens. The test can be used to determine if the middle ear is being ventilated. Sonotubometry results currently have a great deal of background noise from the throat, and recordings are done “in the blind” without any correlation to actual visualized events within the Eustachian tube. With the use of the present invention, a Eustachian tube is recorded by video in the endoscope 44 while an embedded speaker 54 (FIG. 9) in the sleeve 10 emits sounds and a microphone in the ear collects the sound after it has traveled across the Eustachian tube. In the procedure, the sleeve 10 and endoscope 44 combination is inserted in the patient's nostril to reach the Eustachian tube while a microphone is placed in the ear. The signal to noise ratio is greatly improved. The embedded speaker 54 can also include the inflatable balloon 64 to seal in the nasopharyngeal orifice of the Eustachian tube, electromyographic recording electrodes (EMGs) 66, and a pressure/airflow transducer 68 as shown in FIG. 15.

In another procedure, the sleeve 10 and endoscope 44 combination can be used to inject a steroid into tissue to prevent scarring. The embedded instrument 18 is the fluid injection needle 56 that can be manipulated to inject steroids into the tissue site as shown in FIG. 10. The sleeve 10 can be applied to the endoscope 44 and, as described above, the endoscope 44 and sleeve 10 combination can be inserted in the patient to the tissue site of interest, the physician can inject the steroid with the fluid injection needle 56, optionally by telescoping the fluid injection needle 56 out of the instrument entrapment portion 20, and finally, the endoscope 44 and sleeve 10 combination can be removed from the tissue.

The sleeve 10 and endoscope 44 combination can be used to locally filter body cavities. The embedded instrument 18 can be the filtration device 58 as shown in FIG. 11. The sleeve 10 can be applied to the endoscope 44 as described above, the endoscope 44 and sleeve 10 combination can be inserted into the cavity wished to be flushed, the physician can manipulate the filtration device 58 to flush out the cavity (optionally by telescoping the filtration device 58 out of the first passageway), and finally, the endoscope 44 and sleeve 10 combination can be removed from the cavity.

The sleeve 10 and endoscope 44/additional instrument 25 combination can also be used in many other procedures. For example, the combination can be used in laser surgery, cardiovascular surgery, ventricular surgery, or GI surgery by generally following the method of performing a surgical procedure as described above.

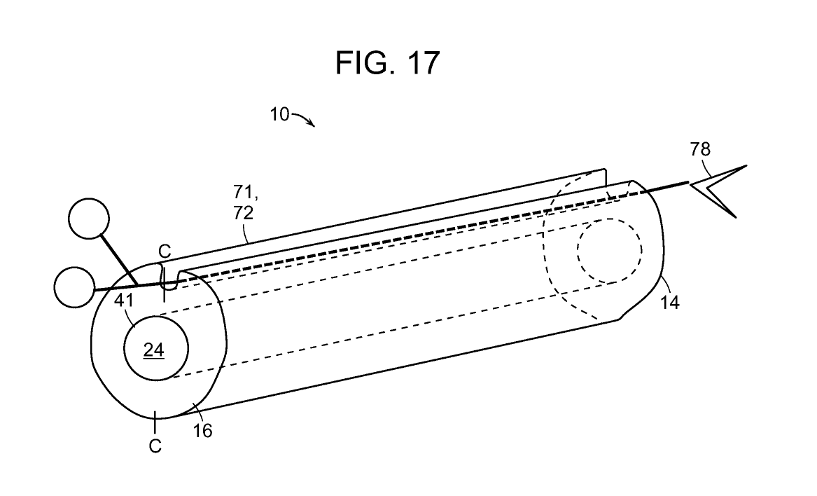

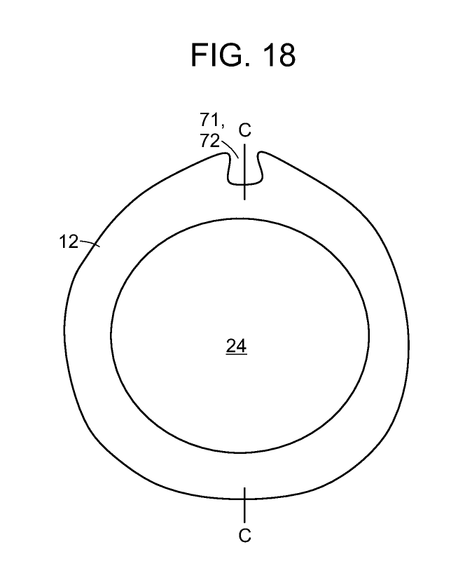

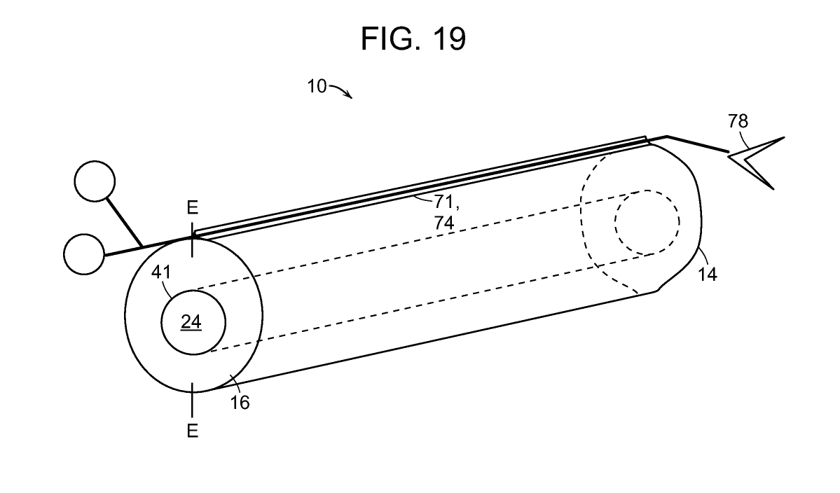

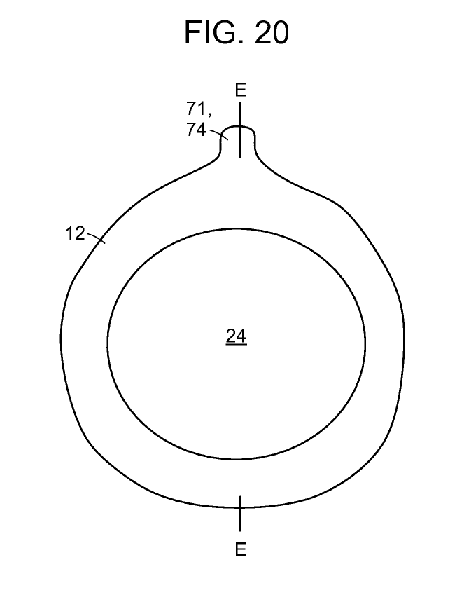

In another embodiment, the sleeve 10 can includes a fixing mechanism 71 such as a longitudinal trough 72 (FIGS. 17 and 18) or a longitudinal ridge 74 (FIGS. 19 and 20) on which a fixed instrument 78 can be fastened. The longitudinal trough 72/ridge 74 extends from the distal end 14 of the body 12 to the proximal end 16 of the body 12. The fixed instrument 78 can be fastened in any suitable way. For example, the trough 72 or the ridge 74 can be shaped to fit the fixed instrument 78. Preferably, the fixed instrument 78 fits snugly against the trough 72/ridge 74. The fixed instrument 78 can be re-used and sterilized before each use. The fixed instrument 78 can be any embedded instruments 18 and can have any of the properties as described above. For example, the fixed instrument 78 can be a needle, a laser, biopsy forceps, a fluid injector, a folding catheter, a dilating probe, a test probe, or any other suitable instrument. The fixed instrument 78 can be used in a similar method as the embedded instruments 18 as described above.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

What is claimed is:

1. A disposable sleeve for covering an instrument comprising a body including a distal end and a proximal end opposite said distal end, said body including an embedded instrument embedded within said body in an instrument entrapment portion defining a longitudinal axis and at least one passageway extending substantially parallel to said longitudinal axis for receiving an additional instrument.

2. The sleeve of claim 1, wherein said embedded instrument includes a functional end and a manipulative end opposite said functional end, said functional end of said embedded instrument extending outwardly from a distal end of said instrument entrapment portion.

3. The sleeve of claim 2, wherein said functional end includes a protruded and a retracted position, wherein said functional end telescopes in and out of said distal end of said instrument entrapment portion to be in said protruded and retracted position, respectively.

4. The sleeve of claim 3, including at least one additional passageway substantially parallel to said first passageway for providing air, suction, or water out of a distal end of said second passageway.

5. The sleeve of claim 2, wherein said passageway includes a sealed distal end for insertion of an endoscope in said passageway, said sealed distal end being a clear material permitting vision through said sealed distal end.

6. The sleeve of claim 5, wherein said functional end of said embedded instrument projects into a field of vision of an endoscope inserted in said passageway.

7. The sleeve of claim 2, wherein said embedded instrument is selected from the group consisting of a folding catheter, a dilating probe, a test probe, a pressure sensing device, a fluid injector, biopsy forceps, a laser, and a needle.

8. The sleeve of claim 2, wherein said body is rigid.

9. The sleeve of claim 2, wherein said body is flexible.

10. The sleeve of claim 2, wherein said sleeve is made from material selected from the group consisting of polytetrafluoroethylene, a flexible plastic, a stiff plastic, a semi-flexible plastic, and combinations thereof.

11. An instrument embedded in a sleeve comprising a body including a distal end and a proximal end, said body including a disposable instrument embedded within said body in an instrument entrapment portion defining a longitudinal axis and at least one passageway extending substantially parallel to said longitudinal axis for receiving an additional instrument.

12. A sleeve comprising a body including an instrument entrapment portion with a disposable instrument embedded therein defining a longitudinal axis, and at least one passageway extending substantially parallel to said longitudinal axis.

13. A disposable sleeve for covering an instrument comprising a body including a distal end and a proximal end opposite said distal end, said body including fixing means defining a longitudinal axis along said body, an instrument fixedly attached to said fixing means along said longitudinal axis, and at least one passageway extending substantially parallel to said longitudinal axis for receiving an additional instrument.

14. The disposable sleeve of claim 13, wherein said fixing means is chosen from the group consisting of a trough and a ridge.

15. A method of covering an instrument with the sleeve of claim 2, including the steps of;
inserting a distal end of an instrument into a proximal end of the passageway in the sleeve;
aligning the distal end of the instrument with the functional end of the embedded instrument while simultaneously aligning a proximal end of the instrument with the manipulative end of the embedded instrument; and
sliding the sleeve over the length of the instrument.

16. A method of performing a medical procedure, including the steps of:
covering an instrument according to the method of claim 15;
inserting the sleeve and instrument combination into the tissue of a patient;
manipulating the functional end of the embedded instrument in the sleeve to perform at least part of the medical procedure; and
removing the instrument and sleeve combination from the patient.

17. The method of claim 16, wherein the covering step is further defined as covering an endoscope.

18. The method of claim 17, further including the step of telescoping the functional end of the embedded instrument into a field of vision of the endoscope after the insertion step.

19. A method of performing sonotubometry, including the steps of:
covering a sonotubometer with a sleeve including an embedded sound recorder according to the method of claim 15;
inserting the sonotubometer and sleeve combination through the nostril of a patient to the Eustachian tube;
recording sound across an Eustachian tube while visualizing the Eustachian tube; and
removing the sonotubometer and sleeve combination from the patient.

20. A method of injecting steroids to prevent scarring, including the steps of:
covering a endoscope with a sleeve including an embedded needle according to the method of claim 15;
inserting the endoscope and sleeve combination through tissue of a patient;
injecting steroids into the tissue; and
removing the endoscope and sleeve combination from the patient.

21. A method of filtrating a cavity, including the steps of:
covering an endoscope with a sleeve including a filtration instrument according to the method of claim 15;
inserting the endoscope and sleeve combination through the tissue of a patient into a cavity;
filtrating the cavity; and
removing the endoscope and sleeve combination from the patient.
